(19) United States
(12) Patent Application Publication
(10) Pub. No.: US 2005/0202514 A1
(43) Pub. Date: Sep. 15, 2005

(54) Competitively Stable Target Cell Populations Isolated from Selectively Expanded Target Cell Populations

(75) Inventor: George Pieczenik, New York, NY (US)

Correspondence Address: DR. GEORGE PIECZENIK, APT. 1F, 412 EAST 55TH STREET, NEW YORK, NY, 10022, US

(73) Assignee: Dr.George Pieczenik, New York, NY (US)

(21) Appl. No.: 10/708,604

(22) Filed: Mar. 15, 2004

(51) Int. Cl. G01N 33/53

(52) U.S. Cl. 435/7.2

(57) ABSTRACT

The invention provides a product and methods of isolating a competitive population from a selective expansion of a predetermined target population of cells, including, inter-alia, the step of (a) reintroducing a selectively amplified population into a starting sample of cells into a growth medium. The invention provides for a competitively stable selectively expanded cell population. In addition, ligands isolated from combinatorial libraries are used as negative and positive cell selectors. A novel coated helical coil reaction vessel is described for continuous and programmable production.

### FEDERAL RESEARCH STATEMENT

[No Federal Funds Were Used in this Invention]

### BACKGROUND OF INVENTION

This invention relates to an improvement for a system for the expansion of targeted cell populations and the isolation of such populations.

Inventor Morey Kraus described a novel and ingenious method for amplifying a target population of cells by predominantly negative selection by the physical removal of undesired non-target cells in U.S. Patent Publication 20020022216 on Feb. 2, 2002. This published application is incorporated herein by reference in its entirety. The continuous growth of both target and non-target cells, with the continuous removal of non-target cells, leads to the amplification of the target cell. This is to be called the Kraus method of Selective Amplification.

However, the Kraus Method does not necessarily create target cells that are stable and competitive to other cell types as they have been primarily isolated by non reiterative negative selection.

This invention, conceived by the inventor during confidential discussions with Morey Kraus, proposes an addition step in the process in order to create a stable and competitive target population as an improvement in the Kraus” Selective Amplification method. This is a reiterative positive selection step. The theory behind this additional step lies in the Lurie-Delbruck fluctuation analysis equations wherein single selected cells appear as “jackpot” amplified populations depending on the selection times relative to the appearance of the resistance to negative selection. The introduction of the re-iteration step allows for the creating of an infinite number of new selection times, thereby, creating a “jackpot” amplification of a targeted cell. A well known cell linage is the hematopoietic cell linage. The hematopoietic stem cell (HSC) is the progenitor of all blood cells and cells in the immune system. Each HSC normally differentiate into a minimum of eight separate blood cell lineages within the myeloid and lymphoid blood cell compartments. One HSC can produce up to fifty million differentiated progeny. The proportion of progenitor to progeny is therefore quite small, unless one introduces some method of amplification. If one could maintain the HSC in a state of dedifferentiation and yet allow HSC”s to continue to be replicated, then a small number of HSCs will become a large pool of HSCs. By re-iteration of a positive and negative selection procedure a stable and competitive population of HSCs (scHSC”s) will appear. This would create a stable and competitive population of scHSC”s then this population would be a definable and useful cellular diagnostic and therapeutic agent and kit.

These scHSCs could then be used to restore or supplement an immune system and/or blood forming system compromised by radiation or chemotherapy, inter alia. Having a large population of scHSCs would be useful as diagnostic and therapeutic agents in the treatment of immune system and/or blood forming disorders. Previous methods of isolating scHSCs and other progenitor stem cells have been selective and not competitive in their isolation procedures. Therefore, the isolated progenitor stem cells were not necessarily stable nor competitive. The additional and novel step suggested in this patent allows for the isolation of scHSCs and other such progenitor cell types, inter alia.

### SUMMARY OF INVENTION

The invention is based on the discovery by Morey Kraus that a predetermined target population of cells, in particular renewable cells, e.g., relatively undifferentiated cells including HSCs, can be clonogenically expanded in a system that either (a) positively selects for cells of the target population, or (b) negatively selects out non-target cells. Selection of this type occurs concurrently with cell growth or intermittently during cell growth. Advantageously, by selectively controlling the relative populations of cells in the system, the invention allows greater expansion of the target population. This selective population control reduces feedback inhibitions, influences factor and substrate consumption rates, and minimizes other limiting factors that tend to occur in conventional batch cultures. In one aspect, the invention features a method of selective expansion of a predetermined target population of cells that includes: (a) introducing a starting sample of cells into a growth medium; (b) causing cells of said predetermined target cell population to divide; and (c) contacting the cells in the growth medium with a selection element, comprising a plurality of selective binding molecules with specific affinity for a predetermined population of cells, so as to select cells of said predetermined target population from other cells in the growth medium (d) re-introducing the isolated target cell back into another starting sample of cells as in step (a) and re-iterating the steps of a-d, until the final target population is both stable and competitive.

The final competitive stable population can be identified by the functional and structural assay that the final proportion of the target cells is approximately equal to the initial proportion re-introduced in the starting sample of cells. The selection may use positive selection (the selective binding molecules are specific for target cells), or negative selection (the selective binding molecules are specific for non-target cells). The improvement to the Kraus Method comprises the preferred embodiment of re-introducing the selectively expanded target cells into a starting population and re-iterating the process until stability and competitiveness is established in the target population.

The invention also features a method of selective expansion of a predetermined target population of cells including: (a) introducing fluid containing a plurality of cells into a growth medium; (b) causing cells of said predetermined target cell population to divide; and (c) selecting cells of said predetermined target population from other cells in the growth medium; wherein steps (b) and (c) are carried out substantially simultaneously and (d) re-introducing the selectively expanded predetermined target cells into the fluid containing a plurality of cells into growth medium and re-iterating steps a-d until a stable and competitive population is achieved. The invention also features a system for continuous selective expansion of a predetermined target population of cells. The system includes (a) a growth medium for supporting cell division; (b) a Helical Coil for receiving said growth medium; and (c) a selection element, the coating in the Helical Coil, positioned to contact said growth medium during or after cell division. The selection element includes a plurality of binding sites bearing a selective binding molecule. The selective binding molecule can have (i) a specific affinity for cells of said predetermined target cell population or (ii) a specific affinity for non-target cells and substantially less affinity for target cells. If desired, the system can further include a reverse selection element having the opposite type of affinity. The selective binding molecules can be peptide ligands isolated from combinatorial peptide libraries. The patents relating to methods and products to produce such binding ligands by the inventor are hereby incorporated by reference. The Pieczenik patents are U.S. Pat. Nos. 5,866,363 and 6,605,448. These binding ligands can be linked to glass with an acrylamide linker with a silane end, inter alia. A coating of binding ligands can be prepared by coating the glass either cylindrical or a helical coil with an amino- acrylic silane or carboxyl-acrylic silane and then binding the ligand either at the amino or carboxyl end with a condensing agent with procedure commonly known in the art. One system of the invention for continuous selective clonogenic expansion of relatively undifferentiated cells includes: (a) a tube containing a plurality of beads of a size which permits a plurality of the undifferentiated cells to grow thereon, the beads bearing on their surfaces a plurality of selective binding molecules capable of binding to a surface antigen present on the relatively undifferentiated cells, wherein the surface antigen is not present on relatively differentiated cells; (b) means for continuously providing nutrients to the relatively undifferentiated cells growing on the beads, wherein the nutrients are delivered via a fluid which flows through the tube and past the beads so that the relatively undifferentiated cells in the tube divide and at least a portion of relatively undifferentiated cells exit the tube with the fluid; and (c) means for continuously harvesting the portion of the relatively undifferentiated cells that exit the tube; and (d) means for re-introducing the output of selected target cells into the input of the selection process; and (e) means for identifying the final stable competitive and amplified target population. The invention can be used to provide competitive and stable stem cells (scHSCs) useful for enhancing the immune system of a patient. The patient's blood or bone marrow is withdrawn (or an allogeneic stem-cell containing sample is provided); stem cells are expanded and harvested according to the invention; and then those cells are re-introduced into the patient, where they will facilitate enhancement or reconstitution of the patient's immune and/or blood forming system. Preferably, the sample taken from the patient is relatively small, e.g., less than about 100 to 200 ml, to minimize trauma to the patient. The preferred potency and dosage of the undifferentiated cells to administer to the patient, and duration of administration, will vary depending upon the condition of the patient's immune or blood forming system, but would generally be expected to be in the range of from about 100 to 1.times.10.sup.6 cells/kg body wgt/dose/day. Alternatively, the invention can be used to provide to a patient a predetermined population of relatively differentiated cells, by providing a sample containing a population of cells which cells are the progenitor to the predetermined population, and using the system of the invention to cause the progenitor cells to proliferate and differentiate to form the predetermined population of cells, e.g., by providing the cells with a growth factor which will cause differentiation. For example, the differentiated cells may be lymphoid precursors, myeloid precursors or erythroid precursors. The invention can also be used to provide to a patient a therapeutic compound produced by a population of cells by using the system of the invention to proliferate cells of the population and to cause the population to produce the substance. The term “continuous,” as used herein, refers to a process which proceeds substantially constantly, with dividing cells being removed from the system shortly after they are born, rather than remaining in culture as in a conventional batch process. This term, as used herein, does not imply that the process is necessarily a steady state process, although in some preferred embodiments steady state may potentially be reached. The term “specific affinity,” as used herein, refers to a tendency to bind a surface molecule or feature that is present on a distinct population of cells and absent on cells not of the population. Examples of such surface molecules or features include but are not limited to cell adhesion molecules, antigens, carbohydrates and functional or non-functional receptors. The term “non-specific interaction,” as used herein, refers to interactions which interfere with and/or reduce the efficiency of desired specific interactions.

The term “competitive” as used herein, refers to the ability of cells to compete in their ability to replicated in competition with one another.

The term “stable,” as used herein, refers to genetic and phenotypic stability of cells. One genotypic and phenotypic characteristic is its ability to compete against similar and dissimilar cell types in various selective and nonselective medium.

The term “combinatorial” and “combinatorial library,” as used herein, refers to mixed peptide libraries that are made either chemically, recombinantly and are contained within scaffolding and are displayed so as to be accessible to binding. Combinatorial libraries of antibodies and of peptides, recombinant or chemical, contain a large numbers of variations around a common binding region in order to offer many possible binding sequences from which binding selection and, thereby, isolation and identification occurs. The utility of the invention and the improvement, thereof, provides tremendous potential for continuous long-term production of cell populations which can be supplied to a patient or other user of the cells almost as soon as the cells are born (or frozen as soon as they are harvested and supplied in frozen form at any desired time). The system can be used as a research tool for studying the effects of biopharmacological agents, growth factors, mitogens and the like, and also as a diagnostic tool, e.g., to gauge the hematopoietic potential of a patient. In addition, another utility of the invention and the improvement, thereof, can be used not only to proliferate relatively undifferentiated cells, but also to produce populations of other cells simply by selecting the appropriate growth factor to supply to the system during expansion, and to produce desired cell by-products, e.g., those which could be administered as therapeutic compounds to a patient. Because the initial cell sample can be autologous, the cell populations or cell by-products produced are likely to be readily accepted by the patient from whom the cell sample was obtained. Because the contents of the system can be frozen, a sample can be taken from a patient, introduced into the system, and then saved for a prolonged period for later use when needed, e.g., when the patient's immune system or blood forming system is challenged.

### BRIEF DESCRIPTION OF DRAWINGS

FIG. 1 is a helical glass coil internally coated with selecting ligands linked by an acrylic silane linker.

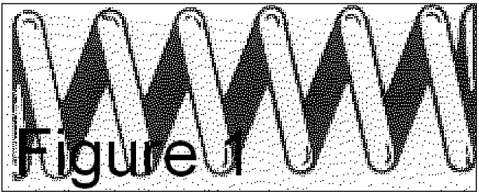

### DETAILED DESCRIPTION

As discussed above, the invention broadly features a method of substantially continuously proliferating cells of a desired target population by providing a system containing a nutrient medium in which cell proliferation can occur, and selecting cells of the target population from non-target cells in the system, concurrently with proliferation, intermittently during proliferation or following proliferation. Cell proliferation and cell selection can be carried out using an almost infinite variety of different techniques and settings, of which only a few are described below by way of example. Many other techniques will be readily perceived by those skilled in the art. All of the preferred techniques, however, are based on the concepts of positive selection (providing a selection element having an affinity for, i.e., “selecting”, target cells) and negative selection (providing a selection element having an affinity for, i.e., “selecting”, non-target cells) and re-iteration to force a competition of selected cells. These two techniques, used alone or in combination, allow unwanted cells to be removed from the system and target cells to be harvested whenever desired. The third technique of reiterating the selected population through the same selection procedure in competition with naive target cells is an additional preferred technique. An example of a negative selection technique can be described as follows. Briefly, one or more silane acrylamide conjugated antibodies which are specific for a predetermined cell population which is not of the predetermined target population is coated on the inside of a glass helical coil. The number of coil turns will determine the specified incubation time the cell suspension has to pass through the helical tubing removing cells not of the predetermined target population from the nutrient medium. For example, ReoPro is an pseudo humanized monoclonal antibody (abciximab) clinically available from Lilly and is an anti-integrin monoclonal antibody. It binds to the glycoprotein IIb/IIIa receptor on the surface of platelets. Coating a helical coil with ReoPro and pumping the growing differentiating culture through the coil will allow the differentiated platelets to be removed from the effluent containing the predetermined progenitor target population. A series of such helical coils coated with various selecting antibodies or legends for differentiated cells will deplete the effluent of differentiated cells. The predetermined target population is collected downstream and returned to the nutrient medium. Such helical glass coils are commercially available in various automated clinical diagnostic pumping apparatus. These pumping apparatus can be used to switch outputs so that they can be programmed to re-introduce the output into the input of the selecting helical coils. These coils can be set up in parallel or in series depending on the selection process, either negative or positive, parallel or sequential necessary for amplification.

An example of a positive selection technique is where one or more selecting ligands for the target cell, antibodies or peptides, inter alia, coat the inside of a glass helical coil. These ligands can also be linked with an acrylic silane linker to the inner surface of the coil. The ligand which is specific for the target cells will coat the inside of the glass helical coil. This coil will bind the target cell and the effluent then is disposed. The target cells are then eluted in a medium containing the identical ligand (without the attaching linker) that was used to bind the target cell as a competitive inhibitor. This competition will force the target cell off the coated inside of the helical coil and allow it to be collected in the effluent. The competitive ligand can be removed by centrifuging the cells out of the solution and decanting the supernatant. This procedure can be modified if one has an antibody which binds the target cell and in which the binding epitopic sequence is known or can be mapped with a combinatorial library. The target cell can then be eluted by the peptide ligand that mimics the antibody's binding epitope.

Various combinations of positive and negative selection helical coils can be arranged as a continuous system in order to amplify and then to compete and stabilize the target cell population and isolate the progenitor cell in an undifferentiated form. Coated Helical Coil Use To use the coated helical coil described above for progenitor stem cell expansion and harvesting a cell proliferation system is connected at one end. Cell proliferation systems include a peristaltic pump to provide flow of fluid through the system, a reagent reservoir, a sampler tube, a waste reservoir, and tubing connecting these components. The cell proliferation system and coated helical coil is placed within an incubator which is maintained at approximately 37 degrees C., 85-90% relative humidity and 5% carbon dioxide throughout the cell proliferation process, inter alia, depending on the requirements of the cell types to be amplified. For another example, to use the Coated Helical Coil, the Helical Coil is to be coated with CD34+ binding antibody or ligand; then a sample containing CD34+ cells, e.g., Ficoll-Paque Gradient Purified Mononuclear Fraction (MNF) (approx. 5.times.10.sup.7 mononuclear cells/ml.) from bone marrow, peripheral or cord blood, or any other source of stem cells, is fed into the Coated Helical Coil through a feed line. Preferably, the peristaltic pump is operated at approximately 1.0 ml./hr. during feed of the sample to the Coated Helical Coil.). The pump should be run in a continuous manner to feed the MNF fraction through the coil and run for a period sufficient to completely feed the MNF and thus saturate the activated sites on the inside of the Coated Helical Coil with CD34 cells.

Once the sample has been fed into the Coated Helical Coil as described in the preceding paragraph, the pump is temporarily shut off while the feed line is connected to reagent reservoir containing a defined nutrient media solution, such as, Iscove's Modified Dulbecco's Medium (IMDM), commercially available from, e.g., GIBCO BRL Products. The pump is then restarted, again preferably at approximately 1 ml/hr, and cell proliferation is allowed to proceed continuously while the nutrient media is fed through the Coated Helical Coil. As soon as substantially all of the activated sites in the coil are saturated, dividing cells will begin to flow out of the coil with the exiting media, to be harvested in the sampler tube (or any other suitable reservoir or conduit).

The use of the Coated Helical Coil described above is in the “continuous” mode of operation. To use the Coated Helical Coil in the “re-iteration” competitive selection mode; it would simply be necessary to provide a conduit to route fluid from the outlet of the coil back to the inlet. Because flowing the fluid through a pump may tend to deleteriously effect the cells, it may be desirable to replace the pump system with a gravity feed system, or otherwise prevent cells from being damaged during recycling. Process Parameters A number of parameters can be varied to affect the rate and purity of the cell output obtained during Coated Helical Coil use. The relationship between dilution rate and cell concentration is described in Principles of Fermentation Technology, P. F. Stanbury & A. Whitaker, Pergammon Press, New York, 1984, at pp. 14-17. The dimensions of the Coated Helical Coil can also be varied. The relationship between coil diameter and the number of coils (the aspect ratio) can be varied to maximize control of process parameters. The volume and purity of the initial sample fed into the coil could also be varied. Reagents Coupling Agents Suitable coupling agents for binding the selective binding molecule to the inner surface of the Helical Coil are those agents that will bind the desired selective binding molecule, but will not bind undesired compounds. When the selective binding molecule is a biotinylated antibody, preferred coupling agents include avidin, streptavidin, NeutrAvidin (commercially available from Pierce Chemical, Rockford, Ill.), and other avidin derivatives. NeutrAvidin is preferred because its pi (isoelectric point) is substantially neutral and thus this protein exhibits very low non-specific binding. Selective ligand peptides synthesized with acrylamite tails can bind directly to an acryl-silane coated inner surface of the coil. Selective Binding Molecules Preferred selective binding molecules are peptide ligands derived from combinatorial libraries. These peptide ligands are designed to mimic the binding capabilities of an Antibody for a particular epitope. Antibodies can be mapped for their epitope specificities. The Pieczenik patents. U.S. Pat. Nos. 5,866,363 and 6,605,448 describe these methods in detail and have been incorporated by reference Suitable antibodies include monoclonal CD34 epitopes and polyclonal CD34 or any uniquely identifiable cell surface antigen or binding site for a desired cell population. Mixtures of antibodies and small peptide ligand mimics of these antibodies can also be used to enhance antibody/cell interactions, both in number and strength of the interactions, which can allow higher flow rates to be used without cells washing off of the beads. Other Reagents A suitable rinse solution to rinse the culture is Dulbecco's PBS, pH 7.4. A suitable rinse solution to rinse the Helical Coil after application of the plasma is Iscoves Modified Dulbecco's Medium (IMDM), which is also used as the nutrient media to promote cell proliferation. Other suitable rinse solutions and nutrient media are known to those skilled in the art. It may be desirable for the nutrient media to be conditioned by cell growth. The level of conditioning of the media can be enhanced by recycling the nutrient media through the chamber while concurrently removing dividing cells from the chamber. Coated Helical Coil Materials The components (coil, tubing, fittings, etc.) should be autoclavable, and preferably also able to withstand gamma irradiation and other harsh methods of sterilization. Moreover, the reactor components should be compatible with tissue culture and should not leach undesirable compounds into the culture medium. The reactor parts further should not accommodate or promote adherence of cells, e.g., by lineage specific antigen receptors, cell adhesion molecules (CAMs) on the cell surface, or secretion products of the cultured cells, unless such antigens, CAMs or secretion products are specifically incorporated into the selection criteria for a given cell proliferation process. Suitable materials that meet these criteria include glass, polypropylene, stainless steel, polytetrafluoroethylene (TEFLON), PFA, and other inert medical grade materials well known in the art. For the tubing, silicone may in some cases be preferred for its relatively high oxygen permeability (allowing sufficient oxygen to reach the cells at lower flow rates); in other cases polytetrafluoroethylene may be preferred for its very low non-specific interaction potential. The fittings which connect the Coated Helical Coil to other elements of the system should be able to accommodate low holdup volume, withstand minimal pressures (typically less than 10 psi), and allow for minimal constriction of flow so as to reduce channeling and adverse fluid flow patterns.

The glass helical coils are preferably borosilicate glass.

Other materials, e.g., polystyrene, or surface activations, e.g., carboxyl, can be used, provided that the surface of the coil is non-porous, to avoid trapping cells or other material in pores on the inner coil surface. The coil surface should also be sufficiently smooth to allow cells, compounds and particulate matter in the chamber to flow past the surface without adhering thereto or diffusing therein. The surface activation can be in the form of reactive groups extending over the inner surface of the coil due to the manner in which the surface has been chemically treated. For example, the coating could be a polypropylene or other polymer and the surface activation could be a cross linked coating, e.g., of an amino acid. The reactive group is selected to be capable of binding the selected coupling agent or, if no coupling agent is used, binding the selective binding molecule itself. The number of binding sites can be varied, however, to suit particular coil dimensions, flow rates, or other process parameters. The bond formed with the reactive group (by the coupling agent or by the selective binding molecule, if no coupling agent is used) is typically covalent. The following section is an example of a therapeutic use for the target cells. The target cells can also be used in many other therapeutic and diagnostic applications. Therapeutic Use A patient requiring immunotherapy would first have a small volume of his or her blood drawn. This blood would used to produce a pool of autologous scHSC's, which would be administered to the patient as an immune system booster prior to a treatment damaging the patient's immune system and/or blood forming system (e.g., chemotherapy), and/or as a stimulant to the patient's compromised immune or blood forming system after the treatment.

1. A method of isolating a stable competitive population of selectively expanded population of a predetermined target population of cells, said method comprising: introducing a starting sample of cells into a growth medium; causing cells of said predetermined target cell population to divide; and contacting the cells in the growth medium with a selection ligand, comprising a plurality of selective binding ligands with specific affinity either for target cells or for a first population of non-target cells, so as to select cells of said predetermined target population from other cells in the growth medium the improvement comprising re-introducing the selectively expanded predetermined target population of cells into a second starting sample of cells and re-iterating the selective expansion method until a stable competitive population is isolated:

2. The competitive population of selectively expanded target cells, wherein, said competitive population of selectively expanded target cells are isolated by the method of claim 1.

3. The selective binding ligand with specific affinity either for target cells or for a first population of non-target cells comprising selective binding ligands isolated from chemical or phage display combinatorial libraries.

4. An internally coated glass helical coil, wherein, said coating comprises a ligand of claim 3 internally linked by a silane acrylic linker to said internally coated glass helical coil.
